(19) United States
(12) Patent Application Publication
(10) Pub. No.: US 2022/0151470 A1
(43) Pub. Date: May 19, 2022

(54) SYSTEMS AND METHODS FOR SEALING A CAMERA MODULE OF AN ENDOSCOPIC IMAGING INSTRUMENT

(71) Applicant: INTUITIVE SURGICAL OPERATIONS, INC, Sunnyvale, CA (US)

(72) Inventors: Viraj A. Patwardhan, Milpitas, CA (US); Matthew M. McConnell, Scotts Valley, CA (US); Parthasarathy Srinivasarajan, Milpitas, CA (US)

(21) Appl. No.: 17/524,183

(22) Filed: Nov. 11, 2021

Related U.S. Application Data
(60) Provisional application No. 63/114,307, filed on Nov. 16, 2020.

(51) Int. Cl. A61B 1/00; A61B 1/05

(52) U.S. Cl. CPC A61B 1/00142; A61B 1/051; A61B 2090/701; A61B 1/0011; A61B 1/00105

(57) ABSTRACT

An endoscopic imaging instrument comprises an optical assembly and a sensor module coupled to the optical assembly. The sensor module comprises a plurality of electronic components and a seal member. The seal member includes an interior surface defining a hermetic cavity. The plurality of electronic components are positioned within the hermetic cavity. The seal member further includes an exterior surface and a wall extending between the interior surface and the exterior surface. The seal member further includes a plurality of connectors within the wall. Each connector of the plurality of connectors is configured to electrically connect with one or more electronic components of the plurality of electronic components.

### CROSS-REFERENCE TO RELATED APPLICATIONS

This application claims the benefit of U.S. Provisional Application 63/114,307 filed Nov. 16, 2020, which is incorporated by reference herein in its entirety.

### FIELD

The present disclosure is directed to systems and methods for sealing a camera module of an endoscopic imaging instrument and more particularly to sealing and protecting electronic components of the camera module.

### BACKGROUND

Minimally invasive medical techniques may generally be intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions an operator may insert minimally invasive medical instruments to reach a target tissue location. Minimally invasive medical tools include instruments such as therapeutic instruments, diagnostic instruments, imaging instruments, and surgical instruments. In some examples, a minimally invasive medical tool may be an imaging instrument, such as a stereoscopic endoscope, for generating three-dimensional images of anatomic areas within a patient anatomy. Imaging instruments may include a camera module, which may include a sensor module with electronic components. Improved systems and methods are needed to seal the electronic components within the sensor module to protect the electronic components during cleaning and sterilization procedures and from fluids and tissue within the patient anatomy.

### SUMMARY

The embodiments of the invention are best summarized by the claims that follow the description.

Consistent with some embodiments, an endoscopic imaging instrument is provided. The endoscopic imaging instrument includes an optical assembly and a sensor module coupled to the optical assembly. The sensor module includes a plurality of electronic components and a seal member. The seal member includes an interior surface defining a hermetic cavity, and the plurality of electronic components are positioned within the hermetic cavity. The seal member further includes an exterior surface and a wall extending between the interior surface and the exterior surface. The seal member further includes a plurality of connectors within the wall. Each connector of the plurality of connectors is configured to electrically connect with one or more electronic components of the plurality of electronic components.

Consistent with other embodiments, a sensor module of an endoscopic imaging instrument is provided. The sensor module includes a plurality of electronic components and a seal member. The seal member includes an interior surface defining a hermetic cavity, and the plurality of electronic components are positioned within the hermetic cavity. The seal member further includes an exterior surface and a wall extending between the interior surface and the exterior surface. The seal member further includes a plurality of connectors within the wall. Each connector of the plurality of connectors is configured to electrically connect with one or more electronic components of the plurality of electronic components.

Consistent with other embodiments, an endoscopic imaging instrument is provided. The endoscopic imaging instrument includes an optical assembly and a sensor module coupled to the optical assembly. The sensor module includes a plurality of electronic components, a seal member, and a connector member. The sensor module further includes a connector seal between the seal member and the connector member. The seal member and the connector seal define a hermetic cavity, and the plurality of electronic components are positioned within the hermetic cavity.

Consistent with other embodiments, a method of manufacturing an imaging instrument is provided. The method includes welding an imaging device to a distal surface of a seal ring. The method further includes brazing a distal surface of an interface component to a proximal surface of the seal ring. The method further includes coupling a plurality of electronic components to a proximal surface of the interface component. The method further includes soldering a distal surface of a seal member to the proximal surface of the interface component. The method further includes, based on the soldering, sealing the plurality of electronic components within a hermetic cavity of the seal member.

Other embodiments include corresponding computer systems, apparatus, and computer programs recorded on one or more computer storage devices, each configured to perform the actions of the methods.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 illustrates a distal end of an imaging instrument according to some examples.

FIG. 2 illustrates a distal end of an imaging instrument including a ceramic seal according to some examples.

FIG. 3A is a cross-sectional perspective illustration of a sensor module, including a ceramic seal, at a distal end of an imaging instrument according to some examples.

FIG. 3B is a cross-sectional illustration of a sensor module, including a ceramic seal, at a distal end of an imaging instrument according to some examples.

FIG. 4A is a perspective illustration of a wall of a ceramic seal of a sensor module at a distal end of an imaging instrument according to some examples.

FIG. 4B is a perspective illustration of an interior cavity of a ceramic seal of a sensor module at a distal end of an imaging instrument according to some examples.

FIG. 5 is a perspective illustration of a sensor module, including a ceramic seal with a strain relief pin, at a distal end of an imaging instrument according to some examples.

FIG. 6 illustrates a distal end of an imaging instrument including a metal seal according to some examples.

FIG. 7A is a cross-sectional perspective illustration of a sensor module, including a metal seal, at a distal end of an imaging instrument according to some examples.

FIG. 7B is a cross-sectional illustration of a sensor module, including a metal seal, at a distal end of an imaging instrument according to some examples.

FIGS. 8A-8B illustrate a distal end of an imaging instrument including a connector with an elastomeric seal according to some examples.

FIGS. 8C-8D illustrate a distal end of an imaging instrument including a connector with a ceramic seal according to some examples.

FIG. 9 is a flowchart illustrating a method of manufacturing an imaging instrument including a seal, according to some examples.

Embodiments of the present disclosure and their advantages are described in the detailed description that follows. It should be appreciated that like reference numerals are used to identify like elements illustrated in one or more of the figures for purposes of illustrating but not limiting embodiments of the present disclosure.

### DETAILED DESCRIPTION

In the following description, specific details describe some embodiments consistent with the present disclosure. Numerous specific details are set forth in order to provide a thorough understanding of the embodiments. It will be apparent to one skilled in the art, however, that some embodiments may be practiced without some or all of these specific details. The specific embodiments disclosed herein are meant to be illustrative but not limiting. One skilled in the art may realize other elements that, although not specifically described, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, components, and circuits have not been described in detail so as not to unnecessarily obscure aspects of the embodiments.

The technology described herein may provide imaging instruments with a sealed camera module. The sealed camera module may protect electronic components of the camera module from various particles, fluids, etc., that may be present within a patient anatomy. The sealed camera module also protects the electronic components from various fluids that may be used to clean and/or sterilize the imaging instrument in one or more cleaning and/or sterilization processes using, for example an autoclave. An autoclave sterilizes the imaging instrument using a combination of steam, low and high pressure, and high temperature. The sealed camera module protects the electronic components from the steam, pressure, and temperature experienced by the imaging instrument during the autoclave cycles. This allows the electronic components to undergo more autoclave cycles than if the imaging instrument did not include the sealed camera module. For example, in some embodiments, the imaging instruments may withstand greater than 20 cycles, greater than 50 cycles, or greater than 100 cycles in an autoclave at a pressure of approximately 34 psi and at a temperature of approximately 138 degrees Celsius for a duration of approximately 18 minutes.

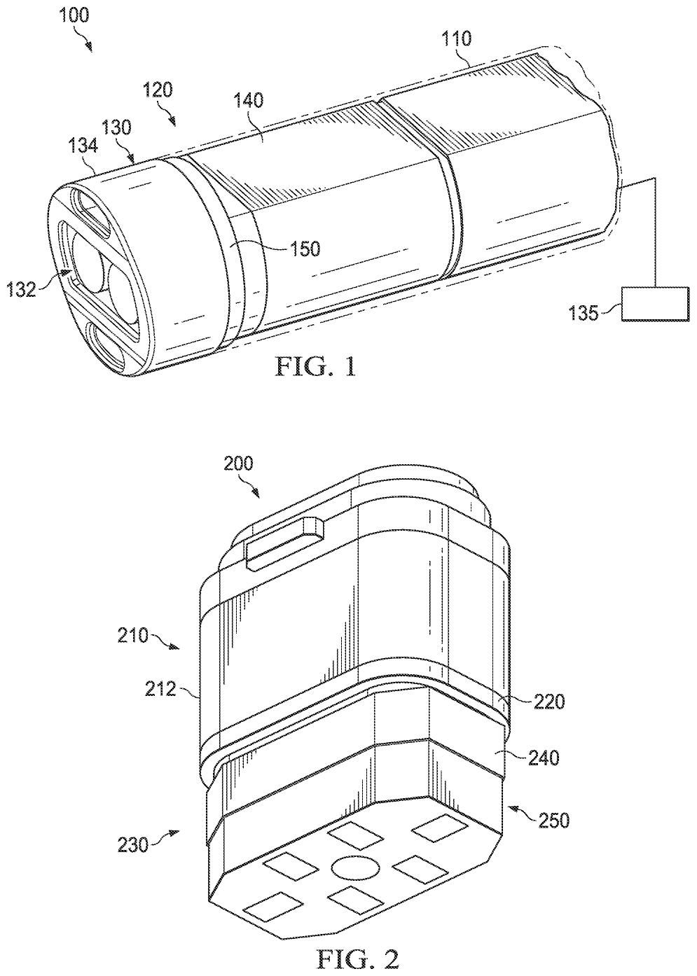

FIG. 1 illustrates an imaging instrument 100 that may be a stereoscopic imaging instrument in some examples. For example, the imaging instrument 100 may be a stereoscopic endoscope. The imaging instrument 100 may include an elongate body 110, an imaging device 130, and a sensor module 140. In some embodiments, the imaging instrument 100 may also include a seal ring 150. In some examples, the imaging device 130, the sensor module 140, and the seal ring 150 may be part of a camera module of the imaging instrument. The imaging device 130 may be coupled to a distal end 120 of the elongate body 110. The seal ring 150 may be coupled to the imaging device 130. The sensor module 140 may also be coupled to the seal ring 150. In some embodiments, the seal ring 150 is positioned between the imaging device 130 and the sensor module 140. The elongate body 110 may be flexible or rigid, and the distal end 120 may be inserted into a patient anatomy to obtain images (e.g., stereoscopic images) of anatomic tissue. In some examples, the distal end 120 may be inserted into an anatomic region such as an abdominal region or a chest region. In some examples, the distal end may be inserted into a natural anatomical passage such as a patient trachea, lung, colon, intestines, stomach, liver, kidneys and kidney calices, brain, heart, circulatory system including vasculature, and/or the like.

In some embodiments, the imaging device 130 may include an optical assembly 132 and a housing 134 enclosing the optical assembly. The optical assembly 132 may include one or more lenses, mirrors, prisms, beamsplitters, windows, filters, or other optical components. In the example of FIG. 1, the housing 134 may extend at least partially into a distal opening of the elongate body 110. In other examples, the housing 134 may extend over or abut to the distal end 120 of the elongate body 110. In some cases, the housing 134 may be coupled to the seal ring 150 (e.g., by welding, soldering, brazing, or the like).

In some examples, the imaging instrument 100 may also include auxiliary systems such as illumination systems, cleaning systems, irrigation systems and/or other systems (not shown) to assist the function of the imaging device 130. In some examples, the imaging instrument 100 may also house cables, linkages, or other steering controls (not shown) to effectuate motion (e.g., pitch and yaw motion) of the distal end 120 of the elongate body 110.

For example, the imaging instrument 100 may be coupled to an imaging control system 135. The imaging control system 135 may include at least one memory and at least one computer processor for effecting control of the imaging instrument 100, including recording image data, sending signals to and receiving information and/or electrical signals from the imaging assembly, operating an auxiliary system, moving the imaging device 130, and/or other functions of the imaging instrument 100. In some embodiments, the imaging control system 135 may be coupled to or be a component of a control system of a robot-assisted medical system. The imaging control system 135 may also include programmed instructions (e.g., a non-transitory machine-readable medium storing the instructions) to implement some or all of the methods described in accordance with aspects disclosed herein.

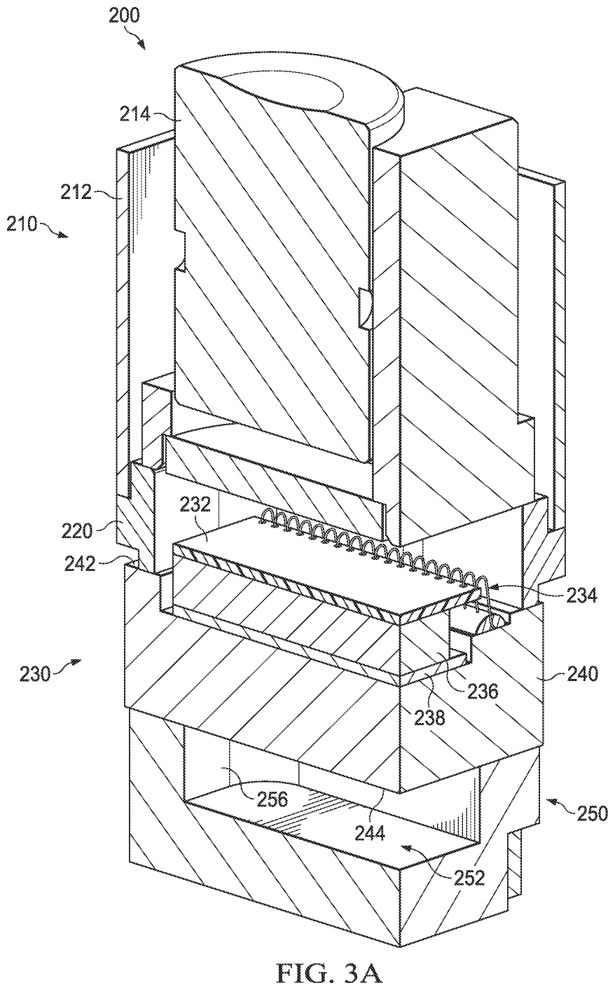

FIG. 2 illustrates a distal end (e.g., the distal end 120) of an imaging instrument 200 (e.g., the imaging instrument 100). The imaging instrument 200 includes an imaging device 210 (e.g., the imaging device 130), a seal ring 220 (e.g., the seal ring 150), and a sensor module 230 (e.g., the sensor module 140). In some examples, the seal ring 220 is coupled to the imaging device 210 and to the sensor module 230 such that the seal ring 220 is positioned between the imaging device 210 and the sensor module 230. The seal ring 220 may be coupled to a housing 212 of the imaging device 210 with, for example a soldered connection, a brazed connection, a welded connection, or the like. In some examples, the housing 212 may contain an optical assembly 214 (e.g. optical assembly 132) and may be formed from a metal (e.g., stainless steel or the like). In some embodiments, the sensor module 230 may include an interface component 240 and a seal member 250. In some examples, the seal ring 220 may be formed from a metal (e.g., stainless steel or the like). In some embodiments, the interface component 240 may be formed from an insulating material, which may be non-conductive and non-porous. For example, the interface component 240 may be formed from a ceramic material. In some cases, the seal member 250 may be formed from a ceramic material.

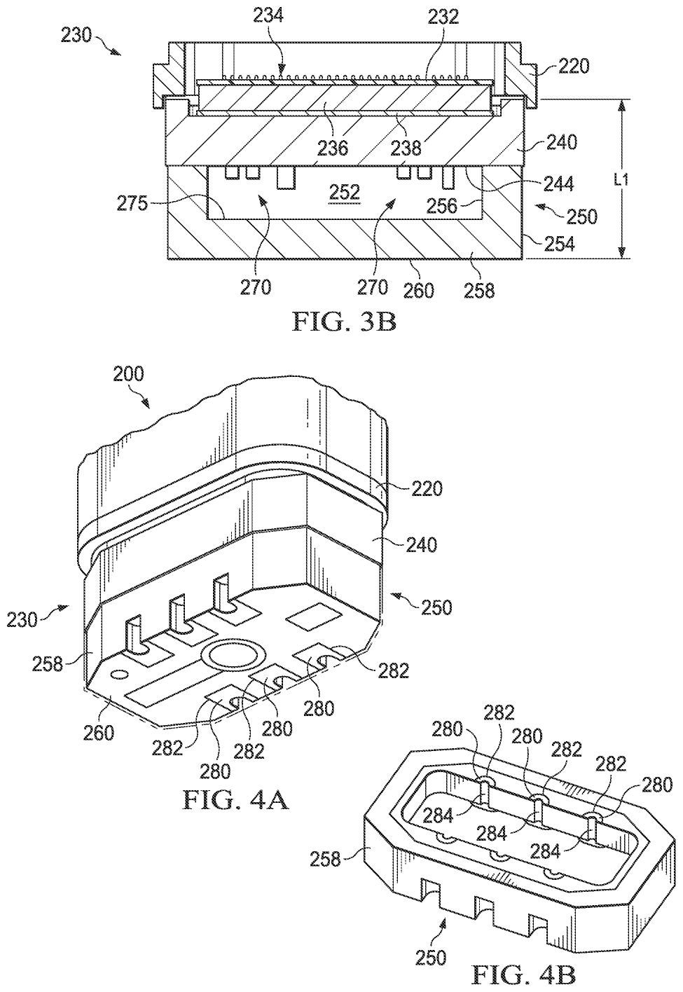

FIG. 3A provides a cross-sectional perspective illustration of the imaging instrument 200. As seen in FIG. 3A, the sensor module 230 includes a sensor 232, one or more electrical connectors (e.g., wires) 234, a spacer 236, a processor 238 (which may be an image sensing processor), the interface component 240, and the seal member 250. In some embodiments, the seal member 250 includes a cavity 252, which may be a hermetic cavity. The sensor 232 may be coupled to the spacer 236, which may be coupled to the processor 238. In some examples, the spacer 236 is positioned between the sensor 232 and the processor 238, as seen in FIG. 3A. The processor 238 may be coupled to the interface component 240 such that the processor 238 is positioned between the spacer 236 and the interface component 240. In some cases, the sensor 232 may be communicatively coupled to the processor 238 via the electrical connectors 234. Thus, data (e.g., sensor data, image data, etc.) may be transferred between the sensor 232 and the processor 238 through the electrical connectors 234. Additionally or alternatively, data may be transferred between the sensor 232 and the processor 238 via a wireless connection.

As further shown in FIG. 3A, a distal surface 242 of the interface component 240 may be coupled to the seal ring 220. The interface component 240 may be coupled to the seal ring 220 via a brazed connection. Additionally or alternatively, the interface component 240 may be coupled to the seal ring 220 via a soldered connection, a welded connection, or any other suitable connection. In some embodiments, a proximal surface 244 of the interface component 240 may be coupled to the seal member 250. The proximal surface 244 may be coupled to the seal member 250 via a soldered connection. Additionally or alternatively, the interface component 240 may be coupled to the seal member 250 via a brazed connection, a welded connection, or any other suitable connection. An interior surface 256 of the seal member 250 may define the hermetic cavity 252. In some embodiments, the hermetic cavity 252 may further be defined by the connection between the interface component 240 and the seal member 250.

FIG. 3B provides a cross-sectional illustration of the sensor module 230. As shown in FIG. 3B, the sensor module 230 may further include one or more electronic components 270. The electronic components 270 may include one or more of a memory, a database, a processor, one or more sensors, etc. The electronic components 270 may be positioned within the hermetic cavity 252. For example, the electronic components 270 may be coupled to the proximal surface 244 of the interface component 240 within the hermetic cavity 252. In some cases, the electronic components 270 may be soldered to the proximal surface 244. The electrical connectors 234 may be connected to the electronic components 270 and to the sensor 232. The electronic components 270 may receive and/or store data (e.g., sensor data, image data, etc.). The electronic components 270 may transfer this data to the sensor 232 and/or may receive this data from the sensor 232 via the electrical connectors 234. Additionally or alternatively, the data may be transferred between the electronic components 270 and the sensor 232 via a wireless connection. In some embodiments, each electronic component 270 is connected to a corresponding electrical connector 234. In alternative embodiments, one electrical connector 234 may connect to all of the electronic components 270. As a further alternative, all of the electrical connectors 234 may connect to each electronic component 270. Any other combination of connections between the electrical connectors 234 and the electronic components 270 may be made.

As further seen in FIG. 3B, the seal member 250 includes an exterior surface 254, an interior surface 256, and a wall 258 extending between the exterior and interior surfaces 254, 256. The seal member 250 may further include a proximal surface 260 (which is part of the exterior surface 254). As discussed above, the interior surface 256 may define the hermetic cavity 252. The hermetic cavity 252 prevents any air, fluid, or other matter from entering the hermetic cavity 252 from an exterior environment 275 surrounding the seal member 250. Because the electronic components 270 are within the hermetic cavity 252, the electronic components 270 are protected from the exterior environment 275. Thus, when the imaging instrument 200 is placed within a patient anatomy, the electronic components 270 are protected from the fluids and other matter present within the patient anatomy. This may allow the life span of the electronic components 270 to be extended. Additionally, this may help reduce the frequency of malfunctions, if any, of the electronic components 270 by preventing contact between the electronic components 270 and any fluids, for example, within the patient anatomy.

Further, the electronic components 270 may be protected from any fluids or products (e.g., steam within an autoclave) that may be used to clean and/or sterilize the imaging instrument 200 in one or more cleaning and/or sterilization processes. The seal member 250 prevents the cleaning/sterilization fluids and/or products from entering the hermetic cavity 252. For example, the seal member 250 may protect the electronic components 270 from various fluids that may be used to clean and/or sterilize the imaging instrument 200 in one or more cleaning and/or sterilization processes using, for example an autoclave. For example, in some embodiments, the imaging instrument 200 may withstand greater than 20 cycles, greater than 50 cycles, or greater than 100 cycles in an autoclave at a pressure of approximately 34 psi and at a temperature of approximately 138 degrees Celsius for a duration of approximately 18 minutes. The imaging instrument 200 may withstand greater than 20 cycles, greater than 50 cycles, or greater than 100 cycles in an autoclave at various other pressures and temperatures for various other durations, as well.

Additionally, the sensor module 230 may have an axial length L1. The axial length L1 may be 1 mm. However, the axial length L1 may be any other suitable length, such as any length from 0.5 mm to 5 mm, for example. A shorter axial length L1 may allow for a larger portion of the imaging instrument 200 to be flexible. For example, as the axial length L1 decreases, the percentage of the overall length of the imaging instrument 200 occupied by the sensor module 230 decreases, and the percentage of the overall length of the imaging instrument 200 occupied by the elongate body increases. Therefore, in examples when the elongate body is flexible, the overall flexibility of the imaging instrument 200 increases as the axial length L1 decreases.

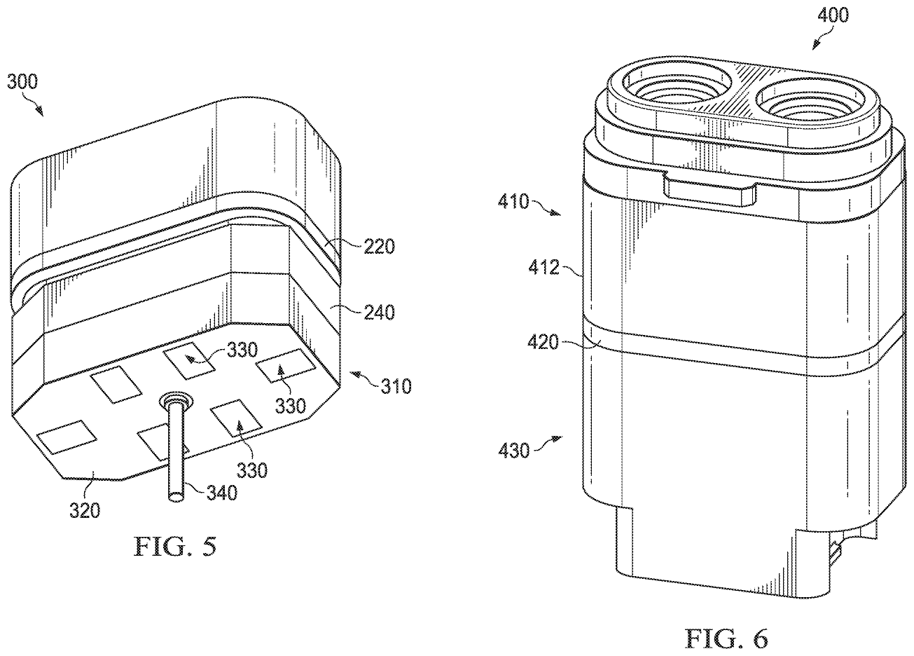

FIGS. 4A and 4B provide illustrations of the seal member 250. For example, FIG. 4B illustrates a perspective view of the seal member 250 illustrating the interior of the wall 258. In some embodiments, one or more connection members 280 may be on the proximal surface 260 of the seal member 250. The connection members 280 may allow for other components (e.g., components proximal to the seal member 250) to connect with the electronic components 270. The connected components may be components of the imaging instrument 200 and/or other components that are not part of the imaging instrument 200, such as one or more of a control system, a teleoperated manipulator, an input control device, and/or the like. In some examples, the connection members 280 are connection pads, connection points, connection surfaces, or the like. Electrical pass-throughs 282 (e.g., castellations, grooves, conduits, etc.) may be included within the wall 258 of the seal member 250. In some embodiments, the electrical connectors 234 may extend from the electronic components 270 through the pass-throughs 282 to the connection members 280. The connection members 280 may extend into the pass-throughs 282.

For example, the connection members 280 each include an inner surface 284 that may be cylindrical, as seen in FIGS. 4A and 4B. In other embodiments, the inner surfaces 284 may be any other suitable shape, such as rectangular, triangular, etc. The connection members 280 further include an outer surface (not shown), which may be cylindrical, rectangular, triangular, or any other suitable shape. In some examples, the outer surface and the inner surface 284 of a particular connection member 280 are different shapes. For example, the outer surface may be rectangular, and the inner surface 284 may be cylindrical.

The outer surface of each connection member 280 may line a corresponding pass-through 282 such that each connection member 280 extends within a corresponding pass-through 282. The shape of the outer surface of each connection member may match the shape of the corresponding pass-through 282 within which each connection member 280 extends. In some cases, the connection members 280 may line some of an axial length or all of the axial length of each corresponding pass-through 282. Similarly, the connection members 280 may line some or all of the interior surface (not shown) of each corresponding pass-through 282. For example, in embodiments where the connection members 280 are half-cylinders, the connection members 280 line some but not all of the interior surface of each corresponding pass-through 282. In embodiments where the connection members 280 are full cylinders, the connection members 280 line all of the interior surface of each corresponding pass-through 282. In some examples, the connection members 280 are formed from a metal (e.g., stainless steel or the like). Additionally or alternatively, the connection members 280 may be gold plated.

In some embodiments, the electrical connectors 234 may extend from the sensor 232 through the pass-throughs 282 to the connection members 280. In some embodiments, each electrical connector 234 extends through a corresponding pass-through 282 and contacts the respective connection member 280. In alternative embodiments, all of the electrical connectors 234 may extend through one pass-through 282. Any other configuration regarding the routing of the electrical connectors 234 through one or more of the pass-throughs 282 may be implemented.

Additionally, while six pass-throughs 282 are shown in FIGS. 4A and 4B, any number of pass-throughs 282 may be included in the wall 258 (e.g., one, two, three, four, five, seven, eight, or any other number of pass-throughs 282). In some embodiments, one or more pass-throughs 282 may extend entirely within the wall 258 such that the pass-throughs 282 are neither open to the cavity 252 nor to the exterior environment surrounding the seal member 250. In some cases, one or more pass-throughs 282 may be partially within the wall 258. For example, one or more pass-throughs 282 may be partially open to the hermetic cavity 252 (as shown in FIG. 4B). In such examples, one or more pass-throughs 282 may form grooves in the interior surface 256 of the wall 258. In some examples, one or more pass-throughs 282 may be partially open to the exterior environment surrounding the seal member 250. In such examples, one or more pass-throughs 282 may form grooves in the exterior surface 254 of the wall 258. In such examples, the pass-throughs 282 do not open to the hermetic cavity 252 to ensure that the hermetic cavity 252 remains hermetically sealed.

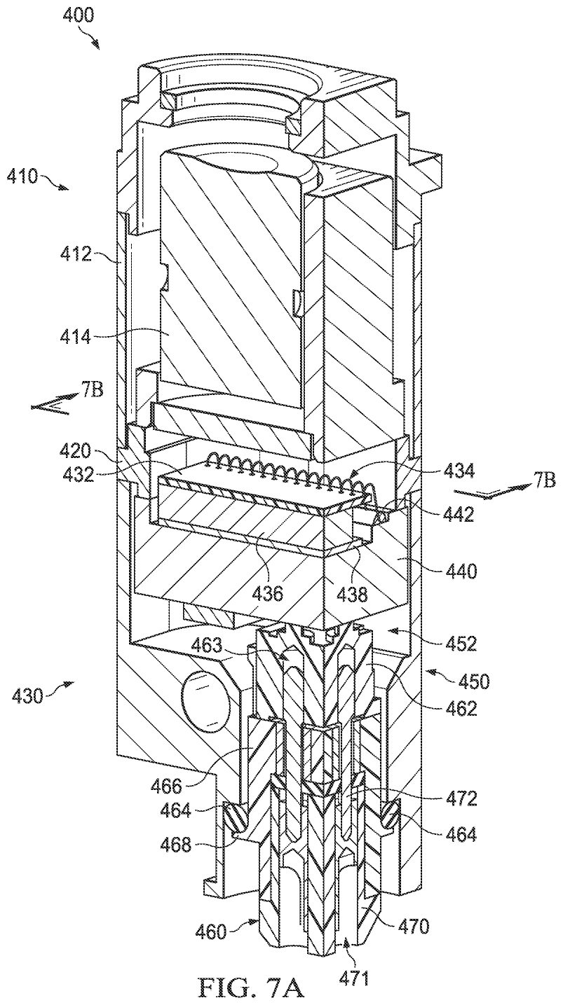

FIG. 5 provides an illustration of an alternative imaging instrument 300 including an alternative seal member 310. The seal member 310 may include like components of the seal member 250 and additional/alternative features, some of which will be discussed below. The seal member 310 includes a proximal surface 320 and one or more connection members 330 (which may be similar to the connection members 280). The seal member 310 may also include a pin 340 extending proximally away from the proximal surface 320. The pin 340 may help reduce strain on one or more of the electrical connectors 234. In some embodiments, the pin 340 may be brazed to the proximal surface 320. In other embodiments, the pin 340 may be coupled to the proximal surface 320 in any other suitable manner (e.g., welding, soldering, etc.).

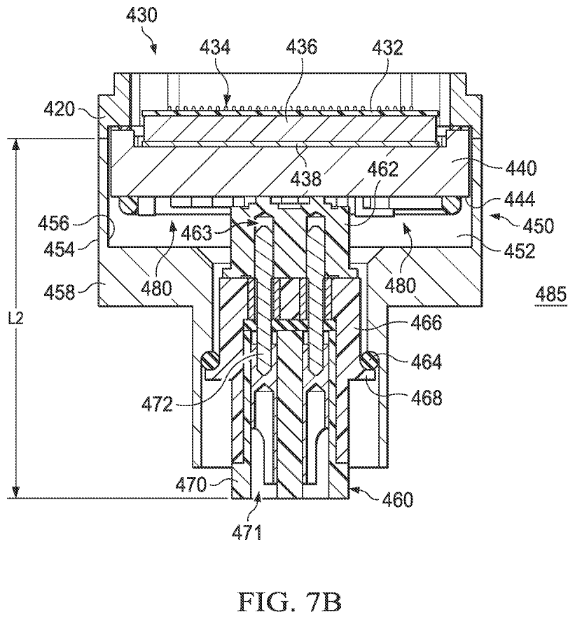

FIG. 6 illustrates a distal end (e.g., the distal end 120) of an imaging instrument 400 (e.g., the imaging instrument 100). The imaging instrument 400 includes an imaging device 410 (e.g., the imaging device 130), a seal ring 420 (e.g., the seal ring 150), and a sensor module 430 (e.g., the sensor module 140). In some examples, the seal ring 420 is coupled to the imaging device 410 and to the sensor module 430 such that the seal ring 420 is positioned between the imaging device 410 and the sensor module 430. The seal ring 420 may be coupled to a housing 412 of the imaging device 410 with, for example, a soldered connection, a brazed connection, a welded connection, or the like. In some examples, the housing 412 may contain an optical assembly 414 (e.g., the optical assembly 132) and may be formed from a metal (e.g., stainless steel or the like). In some embodiments, the seal ring 420 may be formed from a metal (e.g., stainless steel or the like). In some embodiments, the interface component 440 may be formed from an insulating material, which may be non-conductive and non-porous. For example, the interface component 440 may be formed from a ceramic material.

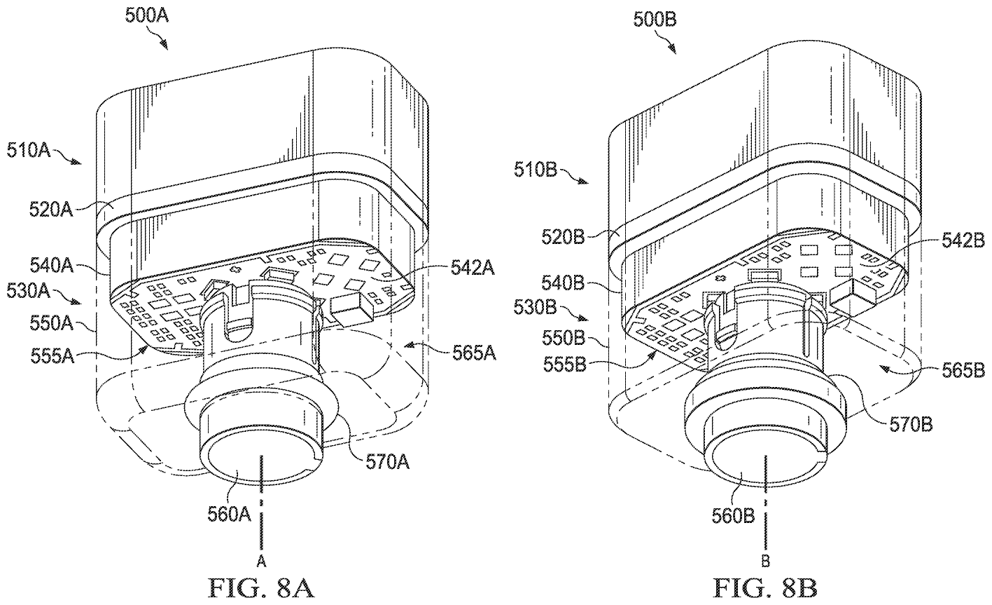

FIG. 7A provides a cross-sectional perspective illustration of the imaging instrument 400. As seen in FIG. 7A, the sensor module 430 includes a sensor 432, one or more electrical connectors (e.g., wires) 434, a spacer 436, a processor 438 (which may be an image sensing processor), an interface component 440, a seal member 450, and a connector member 460. In some embodiments, the seal member 450 may be formed from a metal (e.g., stainless steel or the like). Additionally, the seal member 450 includes a cavity 452, which may be a hermetic cavity. As shown in FIG. 7A, the interface component 440 may extend within the hermetic cavity 452. In some examples, the interface components 440 is entirely housed within the cavity 452. The sensor 432 may be coupled to the spacer 436, which may be coupled to the processor 438. In some examples, the spacer 436 is positioned between the sensor 432 and the processor 438, as seen in FIG. 7A. The processor 438 may be coupled to the interface component 440 such that the processor 438 is positioned between the spacer 436 and the interface component 440. In some cases, the sensor 432 may be coupled to the processor 438 via the electrical connectors 434. Thus, data (e.g., sensor data, image data, etc.) may be transferred between the sensor 432 and the processor 438 through the electrical connectors 434. Additionally or alternatively, data may be transferred between the sensor 432 and the processor 438 via a wireless connection.

As further shown in FIG. 7A, a distal surface of the seal member 450 may be coupled to the seal ring 420. The seal member 450 may be coupled to the seal ring 420 via a welded connection. Additionally or alternatively, the seal member 450 may be coupled to the seal ring 420 via a soldered connection, a brazed connection, or any other suitable connection. In some embodiments, a distal surface 442 of the interface component 440 may be coupled to the seal ring 420. The distal surface 442 may be coupled to the seal ring 420 via a brazed connection. Additionally or alternatively, the interface component 440 may be coupled to the seal ring 420 via a soldered connection, a welded connection, or any other suitable connection.

Additionally, the connector member 460 may include a connector component 462, a seal 464 (e.g., an elastomeric seal, such as an o-ring), a shoulder member 466, a proximal interface member 470, and a distal interface member 472. The connector component 462 may be coupled to the proximal surface 444 of the interface component 440. The seal 464 may be positioned between the shoulder member 466 and the seal member 450 and may create a hermetic seal between these components. In some examples, the seal 464 may rest on a shelf 468 of the shoulder member 466, as shown in FIG. 7A, to create the hermetic seal. An interior surface of the seal member 450 may define the hermetic cavity 452. In some embodiments, the hermetic cavity 452 may further be defined by the connection between the seal member 450 and the seal ring 420 and the seal between the connector member 460 and the seal member 450.

In some embodiments, the proximal interface member 470 is coupled to the distal interface member 472. For example, the distal interface member 472 may fit within one or more channels 471 of the proximal interface member 470. The distal interface member 472 may also be coupled to the connector component 462. For example, the distal interface member 472 may fit within one or more channels 463 of the connector component 462. The shoulder member 466 may extend around the proximal interface member 470, the distal interface member 472, and/or both the proximal and distal interface members 470, 472. The proximal interface member 470 may be coupled to components of the imaging instrument 400 that are proximal to the seal member 450, such as via one or more electronic components 480.

FIG. 7B provides a cross-sectional illustration of the sensor module 430. As shown in FIG. 7B, the sensor module 430 may further include the one or more electronic components 480. The electronic components 480 may include one or more of a memory, a database, a processor, one or more sensors, etc. The electronic components 480 may be positioned within the hermetic cavity 452. For example, the electronic components 480 may be coupled to a proximal surface 444 of the interface component 440 within the hermetic cavity 452. In some cases, the electronic components 480 may be soldered to the proximal surface 444. The electrical connectors 434 may be connected to the electronic components 480 and to the sensor 432. The electronic components 480 may receive and/or store data (e.g., sensor data, image data, etc.). The electronic components 480 may transfer this data to the sensor 432 and/or may receive this data from the sensor 432 via the electrical connectors 434. Additionally or alternatively, the data may be transferred between the electronic components 480 and the sensor 432 via a wireless connection. In some embodiments, each electronic component 480 is connected to a corresponding electrical connector 434. In alternative embodiments, one electrical connector 434 may connect to all of the electronic components 480. As a further alternative, all of the electrical connectors 434 may connect to each electronic component 480. Any other combination of connections between the electrical connectors 434 and the electronic components 480 may be made.

As further seen in FIG. 7B, the seal member 450 includes an exterior surface 454, an interior surface 456, and a wall 458 extending between the exterior and interior surfaces 454, 456. As discussed above, the interior surface 456 may define the hermetic cavity 452. The hermetic cavity 452 prevents any air, fluid, or other matter from entering the hermetic cavity 452 from an exterior environment 485 surrounding the seal member 450. Because the electronic components 480 are within the hermetic cavity 452, the electronic components 480 are protected from the exterior environment 485. Thus, when the imaging instrument 400 is placed within a patient anatomy, the electronic components 480 are protected from the fluids and other matter present within the patient anatomy. This may allow the life span of the electronic components 480 to be extended. Additionally, this may help reduce the frequency of malfunctions, if any, of the electronic components 480 by preventing contact between the electronic components 480 and any fluids, for example, within the patient anatomy.

Further, the electronic components 480 may be protected from any fluids or products (e.g., steam within an autoclave) that may be used to clean and/or sterilize the imaging instrument 400 in one or more cleaning and/or sterilization processes. The seal member 450 prevents the cleaning/sterilization fluids and/or products from entering the hermetic cavity 452. For example, the seal member 450 may protect the electronic components 480 from various fluids that may be used to clean and/or sterilize the imaging instrument 400 in one or more cleaning and/or sterilization processes using, for example an autoclave. For example, in some embodiments, the imaging instrument 400 may withstand greater than 20 cycles, greater than 50 cycles, or greater than 100 cycles in an autoclave at a pressure of approximately 34 psi and at a temperature of approximately 138 degrees Celsius for a duration of approximately 18 minutes. The imaging instrument 400 may withstand greater than 20 cycles, greater than 50 cycles, or greater than 100 cycles in an autoclave at various other pressures and temperatures for various other durations, as well.

Additionally, the sensor module 430 may have an axial length L2. The axial length L2 may be 5 mm. However, the axial length L2 may be any other suitable length, such as any length from 0.5 mm to 10 mm, for example. A shorter axial length L2 may allow for a larger portion of the imaging instrument 400 to be flexible. For example, as the axial length L2 decreases, the percentage of the overall length of the imaging instrument 400 occupied by the sensor module 430 decreases, and the percentage of the overall length of the imaging instrument 400 occupied by the elongate body increases. Therefore, in examples when the elongate body is flexible, the overall flexibility of the imaging instrument 400 increases as the axial length L2 decreases. In some embodiments, the axial length L2 is longer than the axial length L1 discussed above with respect to FIG. 3B. In such embodiments, an imaging instrument that includes the seal member 250 may be more flexible than an imaging instrument that includes the seal member 450.

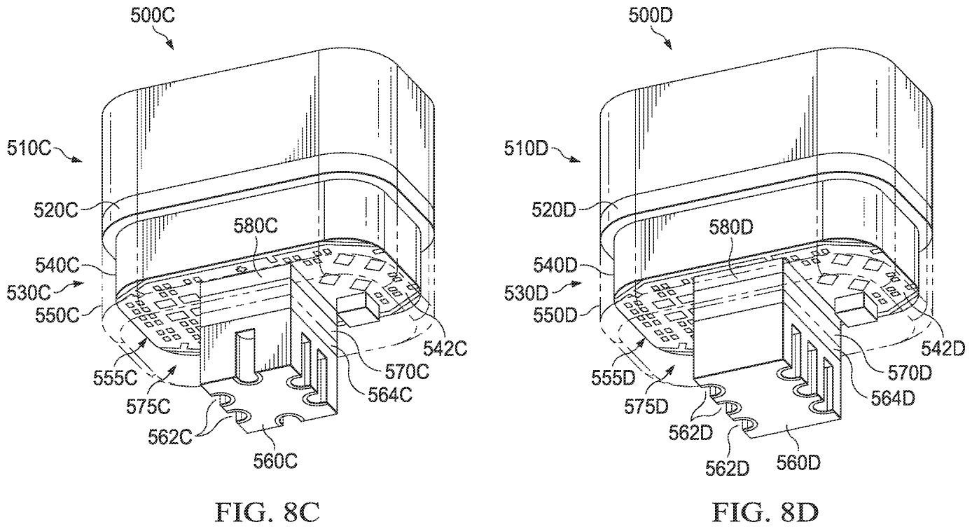

FIG. 8A provides an illustration of an imaging instrument 500A (e.g., the imaging instrument 100). The imaging instrument 500A may include an imaging device 510A (e.g., the imaging device 130), a seal ring 520A (e.g., the seal ring 150), and a sensor module 530A (e.g., the sensor module 140). In some embodiments, the sensor module 530A includes an interface component 540A (e.g., the interface component 440), a housing 550A, a connector 560A, and a seal member 570A. The housing 550A may be coupled to the seal ring 520A with, for example, a soldered connection, a brazed connection, a welded connection, or the like. In some embodiments, the housing 550A may be formed from a metal (e.g., stainless steel or the like). In some cases, the connector 560A may be coupled to a proximal surface 542A of the interface component 540A. The connector 560A may also be coupled to the housing 550A via the seal member 570A. Additionally, the connector 560A may include a longitudinal axis A. In some examples, the seal member 570A may be an elastomeric seal (e.g., an o-ring). The seal member 570A may provide a hermetic cavity 565A within the housing 550A. One or more electronic components 555A may be positioned within the hermetic cavity 565A and may be protected from any matter outside of the hermetic cavity 565A. Additionally, the seal member 570A may be compressed in a radial direction, such as a direction generally perpendicular to the axis A. The compression of the seal member 570A may result in a hermetic seal between the housing 550A and the connector 560A.

FIG. 8B provides an illustration of an imaging instrument 500B (e.g., the imaging instrument 100). The imaging instrument 500B may include an imaging device 510B (e.g., the imaging device 130), a seal ring 520B (e.g., the seal ring 150), and a sensor module 530B (e.g., the sensor module 140). In some embodiments, the sensor module 530B includes an interface component 540B (e.g., the interface component 440), a housing 550B, a connector 560B, and a seal member 570B. The housing 550B may be coupled to the seal ring 520B with, for example, a soldered connection, a brazed connection, a welded connection, or the like. In some embodiments, the housing 550B may be formed from a metal (e.g., stainless steel or the like). In some cases, the connector 560B may be coupled to a proximal surface 542B of the interface component 540B. The connector 560B may also be coupled to the housing 550B via the seal member 570B. Additionally, the connector 560B may include a longitudinal axis B. In some examples, the seal member 570B may be an elastomeric seal (e.g., an o-ring). The seal member 570B may provide a hermetic cavity 565B within the housing 550B. One or more electronic components 555B may be positioned within the hermetic cavity 565B and may be protected from any matter outside of the hermetic cavity 565B. Additionally, the seal member 570B may be compressed in an axial direction, such as a direction generally parallel to the axis B. The compression of the seal member 570B may result in a hermetic seal between the housing 550B and the connector 560B.

FIG. 8C provides an illustration of an imaging instrument 500C (e.g., the imaging instrument 100). The imaging instrument 500C may include an imaging device 510C (e.g., the imaging device 130), a seal ring 520C (e.g., the seal ring 150), and a sensor module 530C (e.g., the sensor module 140). In some embodiments, the sensor module 530C includes an interface component 540C (e.g., the interface component 440), a housing 550C, a connector 560C, a seal member 570C, and a coupling member 580C. The housing 550C may be coupled to the seal ring 520C with, for example, a soldered connection, a brazed connection, a welded connection, or the like. In some embodiments, the housing 550C may be formed from a metal (e.g., stainless steel or the like).

In some cases, the connector 560C may include electrical pass-throughs 562C (which are substantially similar to the electrical pass-throughs 282 discussed above with respect to FIGS. 4A-4B). As shown in FIG. 8C, at least one pass-through 562C may be included in each side of the connector 560C. Additionally, one or more sides of the connector 560C may include more than one pass-through 562C. In some examples, the connector 560C may further include a seal layer 564C. The seal layer 564C may be coupled to the seal member 570C with, for example, a soldered connection, a brazed connection, a welded connection, or the like. In some cases, the seal member 570C may be coupled to the coupling member 580C with, for example, a soldered connection, a brazed connection, a welded connection, or the like. The seal member 570C may provide a hermetic cavity 575C within the housing 550C. One or more electronic components 555C may be positioned within the hermetic cavity 575C and may be protected from any matter outside of the hermetic cavity 575C.

FIG. 8D provides an illustration of an imaging instrument 500D (e.g., the imaging instrument 100). The imaging instrument 500D may include an imaging device 510D (e.g., the imaging device 130), a seal ring 520D (e.g., the seal ring 150), and a sensor module 530D (e.g., the sensor module 140). In some embodiments, the sensor module 530D includes an interface component 540D (e.g., the interface component 440), a housing 550D, a connector 560D, a seal member 570D, and a coupling member 580D. The housing 550D may be coupled to the seal ring 520D with, for example, a soldered connection, a brazed connection, a welded connection, or the like. In some embodiments, the housing 550D may be formed from a metal (e.g., stainless steel or the like).

In some cases, the connector 560D may include electrical pass-throughs 562D (which are substantially similar to the electrical pass-throughs 282 discussed above with respect to FIGS. 4A-4B). As shown in FIG. 8D, the pass-throughs 562D may be included in at least two sides of the connector 560D. Additionally, one or more of the sides may include more than one pass-through 562D. In some examples, the connector 560D may further include a seal layer 564D. The seal layer 564D may be coupled to the seal member 570D with, for example, a soldered connection, a brazed connection, a welded connection, or the like. In some cases, the seal member 570D may be coupled to the coupling member 580D with, for example, a soldered connection, a brazed connection, a welded connection, or the like. The seal member 570D may provide a hermetic cavity within the housing 550D. One or more electronic components 555D may be positioned within the hermetic cavity 575D and may be protected from any matter outside of the hermetic cavity 575D.

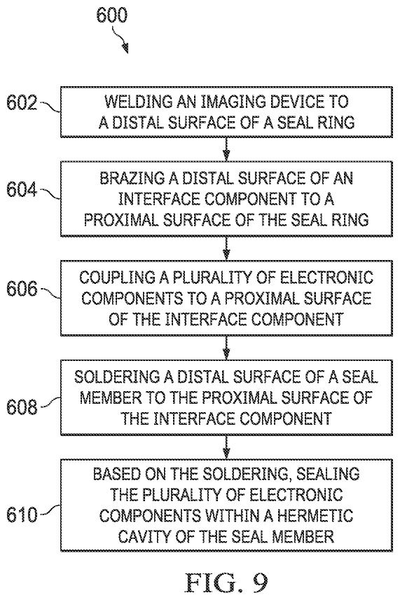

FIG. 9 is a flowchart illustrating an example method 600 for manufacturing an imaging instrument, including any of those previously described. The method 600 is illustrated as a set of operations or processes 602 through 610. The processes illustrated in FIG. 9 may be performed in a different order than the order shown in FIG. 9, and one or more of the illustrated processes might not be performed in some embodiments of the method 600. Additionally, one or more processes that are not expressly illustrated in FIG. 9 may be included before, after, in between, or as part of the illustrated processes.

At a process 602, an imaging device (e.g., the imaging device 210) is welded to a distal surface of a seal ring (e.g., the seal ring 220). The seal ring may be formed from a metal (e.g., stainless steel or the like).

At a process 604, the proximal surface of the seal ring is brazed to a distal surface of an interface component (e.g., the interface component 240). In some embodiments, the interface component may be formed from an insulating material, which may be non-conductive and non-porous. For example, the interface component may be formed from a ceramic material.

At a process 606, one or more electronic components (e.g., the electronic components 270) are coupled to a proximal surface of the interface component. The electronic components may include one or more of a memory, a database, a processor, one or more sensors, etc.

At a process 608, a distal surface of a seal member (e.g., the seal member 250) is soldered to the proximal surface of the interface component. In some embodiments, the seal member may be formed from an insulating material, which may be non-conductive and non-porous. For example, the seal member may be formed from a ceramic material.

At a process 610, as a result of the soldering, the plurality of electronic components are sealed within a hermetic cavity (e.g., the hermetic cavity 252) of the seal member. The hermetic cavity may be defined by an interior surface of the seal member. The hermetic cavity may also be defined by the soldered joint between the interface component and the seal member.

In the description, specific details have been set forth describing some embodiments. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure.

The singular forms “a”, “an”, and “the” are intended to include the plural forms as well, unless the context indicates otherwise. And the terms “comprises,” “comprising,” “includes,” “has,” and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components. The auxiliary verb “may” likewise implies that a feature, step, operation, element, or component is optional.

Elements described in detail with reference to one embodiment, implementation, or application optionally may be included, whenever practical, in other embodiments, implementations, or applications in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions.

A computer is a machine that follows programmed instructions to perform mathematical or logical functions on input information to produce processed output information. A computer includes a logic unit that performs the mathematical or logical functions, and memory that stores the programmed instructions, the input information, and the output information. The term “computer” and similar terms, such as “processor” or “controller” or “control system”, are analogous.

Although some of the examples described herein refer to surgical procedures or instruments, or medical procedures and medical instruments, the techniques disclosed apply to non-medical procedures and non-medical instruments. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy), and performing procedures on human or animal cadavers. Further, these techniques can also be used for surgical and nonsurgical medical treatment or diagnosis procedures.

Further, although some of the examples presented in this disclosure discuss teleoperational robotic systems or remotely operable systems, the techniques disclosed are also applicable to computer-assisted systems that are directly and manually moved by operators, in part or in whole.

Additionally, one or more elements in embodiments of this disclosure may be implemented in software to execute on a processor of a computer system such as a control processing system. When implemented in software, the elements of the embodiments of the present disclosure are essentially the code segments to perform the necessary tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc.

Note that the processes and displays presented may not inherently be related to any particular computer or other apparatus, and various systems may be used with programs in accordance with the teachings herein. The required structure for a variety of the systems discussed above will appear as elements in the claims. In addition, the embodiments of the present disclosure are not described with reference to any particular programming language. It will be appreciated that a variety of programming languages may be used to implement the teachings of the present disclosure as described herein.

While certain example embodiments of the present disclosure have been described and shown in the accompanying drawings, it is to be understood that such embodiments are merely illustrative of and not restrictive to the broad disclosed concepts, and that the embodiments of the present disclosure not be limited to the specific constructions and arrangements shown and described, since various other modifications may occur to those ordinarily skilled in the art.

What is claimed is:

1. An endoscopic imaging instrument comprising:
an optical assembly; and
a sensor module coupled to the optical assembly, the sensor module comprising:
a plurality of electronic components; and
a seal member including:
an interior surface defining a hermetic cavity, wherein the plurality of electronic components are positioned within the hermetic cavity;
an exterior surface;
a wall extending between the interior surface and the exterior surface; and
a plurality of connectors within the wall, wherein each connector of the plurality of connectors is configured to electrically connect with one or more electronic components of the plurality of electronic components.

2. The endoscopic imaging instrument of claim 1, wherein the sensor module further comprises an interface component coupled to the seal member.

3. The endoscopic imaging instrument of claim 2, wherein the plurality of electronic components are coupled to a proximal surface of the interface component.

4. The endoscopic imaging instrument of claim 2, wherein the seal member is brazed to the interface component.

5. The endoscopic imaging instrument of claim 2, wherein the seal member is ceramic, and wherein the interface component is ceramic.

6. The endoscopic imaging instrument of claim 2, wherein the sensor module further includes a seal ring, and wherein the optical assembly includes a housing coupled to the seal ring.

7. The endoscopic imaging instrument of claim 1, wherein each electronic component of the plurality of electronic components includes a corresponding conduit extending through the wall of the seal member.

8. A sensor module of an endoscopic imaging instrument, the sensor module comprising:
a plurality of electronic components; and
a seal member including:
an interior surface defining a hermetic cavity, wherein the plurality of electronic components are positioned within the hermetic cavity;
an exterior surface;
a wall extending between the interior surface and the exterior surface; and
a plurality of connectors within the wall, wherein each connector of the plurality of connectors is configured to electrically connect with one or more electronic components of the plurality of electronic components.

9. The sensor module of claim 8, further comprising an interface component coupled to the seal member.

10. The sensor module of claim 9, wherein the plurality of electronic components are coupled to a proximal surface of the interface component.

11. The sensor module of claim 9, wherein the seal member is brazed to the interface component.

12. The sensor module of claim 10, wherein the seal member is ceramic, and wherein the interface component is ceramic.

13. The sensor module of claim 10, further comprising a seal ring coupled to a housing of an optical assembly of the endoscopic imaging instrument.

14. The sensor module of claim 8, wherein each electronic component of the plurality of electronic components includes a corresponding conduit extending through the wall of the seal member.

15. An endoscopic imaging instrument comprising:
an optical assembly; and
a sensor module coupled to the optical assembly, the sensor module comprising:
a plurality of electronic components;
a seal member;
a connector member; and
a connector seal between the seal member and the connector member,
wherein the seal member and the connector seal define a hermetic cavity, and wherein the plurality of electronic components are positioned within the hermetic cavity.

16. The endoscopic imaging instrument of claim 15, wherein the sensor module further comprises an interface component and a seal ring, the seal ring coupled to the seal member.

17. The endoscopic imaging instrument of claim 16, wherein the plurality of electronic components are coupled to a proximal surface of the interface component.

18. The endoscopic imaging instrument of claim 16, wherein the interface component is positioned within the hermetic cavity.

19. The endoscopic imaging instrument of claim 16, wherein the seal member is welded to the seal ring.

20. The endoscopic imaging instrument of claim 16, wherein the seal member is metal, and wherein the seal ring is metal.
